(19) United States
(12) Patent Application Publication
(10) Pub. No.: US 2025/0312029 A1
(43) Pub. Date: Oct. 9, 2025

(54) SURGICAL IMPLANT ASSEMBLY

(71) Applicant: Field Orthopaedics PTY Ltd, Red Hill (AU)

(72) Inventors: Greg COUZENS, Red Hill (AU); Kau Shean LIANG, Red Hill (AU); Michael MAURER, Red Hill (AU)

(73) Assignee: Field Orthopaedics PTY Ltd, Red Hill (AU)

(21) Appl. No.: 19/243,113

(22) Filed: Jun. 19, 2025

Related U.S. Application Data
(63) Continuation-in-part of application No. 19/083,583, filed on Mar. 19, 2025.
Application No. 19/083,583 is a continuation of application No. PCT/AU2024/050418, filed on Apr. 30, 2024.

(30) Foreign Application Priority Data
May 5, 2023 (AU) 2023901361

(51) Int. Cl. A61B 17/04

(52) U.S. Cl. CPC A61B 17/0401; A61B 2017/0414; A61B 2017/042

(57) ABSTRACT

A surgical implant assembly comprising: first and second anchor bodies adapted to engage respective bone surfaces; a primary suture having a suture length extending between first and second terminal segments of the primary suture, the suture length of the primary suture further comprising: a first suture segment that passes through the first anchor body; and a second suture segment passing through the second anchor body with a bridging segment of the primary suture extending between the first and second suture segments of the primary suture wherein the first suture segment of the primary suture lies in between the bridging segment and the first terminal segment of the primary suture and wherein the second suture segment of the primary suture lies in between the bridging segment and the second terminal segment of the primary suture; a secondary suture having a suture length extending between first and second terminal segments of the secondary suture, the suture length of the secondary suture further comprising: a first suture segment that passes through the first anchor body; and a second suture segment passing through the second anchor body with a bridging segment of the secondary suture extending between the first and second suture segments of the primary suture wherein the first suture segment of the secondary suture lies in between the bridging segment and the first terminal segment of the secondary suture and wherein the second suture segment of the secondary suture lies in between the bridging segment and the second terminal segment of the secondary suture; and wherein during use, the bridging segment of the primary suture is adapted to overlap with the bridging segment of the secondary suture.

### RELATED APPLICATIONS

This application is a Continuation-in-Part (CIP) of U.S. patent application Ser. No. 19/083,583 filed on Mar. 19, 2025, which is a Continuation of PCT Patent Application No. PCT/AU2024/050418 having international filing date of Apr. 30, 2024, which claims the benefit of priority of Australia Patent Application No. 2023901361 filed on May 5, 2023. The contents of the above applications are all incorporated by reference as if fully set forth herein in their entirety.

### FIELD AND BACKGROUND OF THE INVENTION

The present invention relates an implantable sling and anchor assembly in combination with suture that is particularly well suited for the surgical repair and reconstruction of joints, preferably of the hands, in particular the basal joint.

Any references to methods, apparatus or documents of the prior art are not to be taken as constituting any evidence or admission that they formed, or form part of the common general knowledge.

Degenerative osteoarthritis of the first carpal-metacarpal joint (basal joint) of the thumb is a common, disabling condition. This condition occurs when the cartilage in the carpometacarpal (CMC) joint wears away. As arthritis progresses, patients suffer from load-related pain, affecting pinching and gripping activities, and experience weakness, severe dysfunction, reduced mobility, and loss of functionality. Trapeziectomy is a common form of surgery which involves a complete removal of the trapezium bone. Some surgeons fill the gap left by the removed bone using a piece of tendon from your wrist. To fill the space left, you may be given an artificial metal and plastic joint, or a silicone rubber spacer. This method involves essentially removing the arthritic articulation by excising the trapezium and temporarily pinning the joint in distraction. This procedure can provide pain relief, but is associated with persistent collapse deformity, proximal migration, and weakness. The key principles of successful basal joint arthroplasty involve trapezial excision, which is required for pain relief, and some form of ligament reconstruction, which restores the function of the important volar stabilizing ligament, thereby re-creating the proper balance, alignment, longitudinal length, and function of the thumb. With ligament reconstruction, not only is pain relief attained but strength, alignment and stability are achieved. While trapeziectomy is one of the leading methods of addressing such arthritis, it is desirable to provide a method and implantable assembly which can simplify the actual surgical procedure and preferably reduce recovery and rehabilitation times after surgery. Long term results with existing trapeziectomy methods have been excellent with regard to pain relief, but the healing process is lengthy, incisions are extensive and multiple, subsidence with loss of arthroplasty space has been reported, pin tract complications do occur, and the technique is technically demanding, lengthy and invariably requires sacrificing a normal, functioning wrist flexor tendon. The present invention aims to address some of these issues without compromising on the excellent results in relation to pain relief.

### SUMMARY OF THE INVENTION

In a first aspect, the invention provides a surgical implant assembly comprising:
first and second anchor bodies adapted to engage respective bone surfaces;
a primary suture having a suture length extending between first and second terminal segments of the primary suture, the suture length of the primary suture further comprising:
   a first suture segment that passes through the first anchor body; and
   a second suture segment passing through the second anchor body with a bridging segment of the primary suture extending between the first and second suture segments of the primary suture wherein the first suture segment of the primary suture lies in between the bridging segment and the first terminal segment of the primary suture and
   wherein the second suture segment of the primary suture lies in between the bridging segment and the second terminal segment of the primary suture;
a secondary suture having a suture length extending between first and second terminal segments of the secondary suture, the suture length of the secondary suture further comprising:
   a first suture segment that passes through the first anchor body; and
   a second suture segment passing through the second anchor body with a bridging segment of the secondary suture extending between the first and second suture segments of the primary suture
   wherein the first suture segment of the secondary suture lies in between the bridging segment and the first terminal segment of the secondary suture and wherein the second suture segment of the secondary suture lies in between the bridging segment and the second terminal segment of the secondary suture; and
      wherein during use, the bridging segment of the primary suture is adapted to overlap with the bridging segment of the secondary suture.

In another aspect, the invention provides a method of performing surgery, the method comprising: providing a surgical implant assembly comprising:
first and second anchor bodies adapted to engage respective bone surfaces;
a primary suture having a suture length extending between first and second terminal segments of the primary suture, the suture length of the primary suture further comprising:
   a first suture segment that passes through the first anchor body; and
   a second suture segment passing through the second anchor body with a bridging segment of the primary suture extending between the first and second suture segments of the primary suture
   wherein the first suture segment of the primary suture lies in between the bridging segment and the first terminal segment of the primary suture and wherein the second suture segment of the primary suture lies in between the bridging segment and the second terminal segment of the primary suture;
a secondary suture having a suture length extending between first and second terminal segments of the primary suture, the suture length of the secondary suture further comprising:
   a first suture segment that passes through the first anchor body; and
   a second suture segment passing through the second anchor body with a bridging segment of the secondary suture extending between the first and second suture segments of the primary suture
   wherein the first suture segment of the secondary suture lies in between the bridging segment and the first terminal segment of the secondary suture and wherein the second suture segment of the secondary suture lies in between the bridging segment and the second terminal segment of the secondary suture; and
      inserting the first anchor body through a first bore drilled across a first bone region such that the first bore extends between proximal end and a distal end of the first bore such that the first anchor body is inserted into an opening at the proximal end and passed through the first bore to position the first anchor body at the distal end of the first bore;
      inserting the second anchor body though a second bore drilled across a second bone region such that the second bore extends between proximal end and a distal end of the bore such that the second anchor body is inserted into an opening at the proximal end of the second bore to position the second anchor body at the distal end of the second bore;
      tensioning the primary suture to effect engagement and initial anchoring of the first and second anchor bodies at the respective distal ends of the first and second bores resulting in the bridging segment of the primary suture to overlap with the bridging segment of the secondary suture and lie along portions of the first and second bone regions;
      tying the terminal ends of tensioned primary suture to form a first knot;
      tensioning the secondary suture to effect further engagement and anchoring of the first and second anchor bodies at the respective distal end of the first and second bores;
      passing the terminal ends of the secondary suture through soft tissue adjoining the first and second bone regions; and
      tying the terminal ends of tensioned secondary suture to form a second knot.

In a second aspect, there is provided a surgical implant assembly comprising:
first and second anchor bodies adapted to engage respective bone surfaces;
a primary suture having a suture length extending between first and second terminal segments of the primary suture, the suture length of the primary suture further comprising:
   a first suture segment that passes through the first anchor body; and
   a second suture segment passing through the second anchor body with a bridging segment of the primary suture extending between the first and second suture segments of the primary suture
   wherein the first suture segment of the primary suture lies in between the bridging segment and the first terminal segment of the primary suture and wherein the second suture segment of the primary suture lies in between the bridging segment and the second terminal segment of the primary suture;
additional secondary sutures comprising a first secondary suture for engaging the first anchor body and a second secondary suture for engaging the second anchor body wherein each of the secondary sutures comprise a suture length extending between first and second terminal segments of the secondary suture and wherein:
the first secondary suture passes through the first anchor body such that no section of the first secondary suture passes through the second anchor body; and
the second secondary suture passes through the second anchor body such that no section of the second secondary suture passes through the first anchor body.

In another aspect, there is provided a method of performing surgery, the method comprising: providing a surgical implant assembly comprising:
first and second anchor bodies adapted to engage respective bone surfaces;
a primary suture having a suture length extending between first and second terminal segments of the primary suture, the suture length of the primary suture further comprising:
a first suture segment that passes through the first anchor body; and
a second suture segment passing through the second anchor body with a bridging segment of the primary suture extending between the first and second suture segments of the primary suture
wherein the first suture segment of the primary suture lies in between the bridging segment and the first terminal segment of the primary suture and wherein the second suture segment of the primary suture lies in between the bridging segment and the second terminal segment of the primary suture;
additional secondary sutures comprising a first secondary suture for engaging the first anchor body and a second secondary suture for engaging the second anchor body wherein each of the secondary sutures comprise a suture length extending between first and second terminal segments of the secondary suture and wherein:
the first secondary suture passes through the first anchor body such that no section of the first secondary suture passes through the second anchor body; and
the second secondary suture passes through the second anchor body such that no section of the second secondary suture passes through the first anchor body
   inserting the first anchor body through a first bore drilled across a first bone region such that the first bore extends between proximal end and a distal end of the first bore such that the first anchor body is inserted into an opening at the proximal end and passed through the first bore to position the first anchor body at the distal end of the first bore;
   inserting the second anchor body though a second bore drilled across a second bone region such that the second bore extends between proximal end and a distal end of the bore such that the second anchor body is inserted into an opening at the proximal end of the second bore to position the second anchor body at the distal end of the second bore;
   tensioning the primary suture to effect engagement and initial anchoring of the first and second anchor bodies at the respective distal ends of the first and second bores resulting in the bridging segment of the primary suture to lie along portions of the first and second bone regions;
   tying the terminal ends of tensioned primary suture to form a primary knot;
   tensioning the first and second secondary sutures to effect further engagement and anchoring of the first and second anchor bodies at the respective distal end of the first and second bores;
   passing the terminal ends of the first and second secondary sutures through soft tissue adjoining the first and second bone regions; and
   tying the terminal ends of the tensioned first and second secondary sutures respectively to form secondary knots.

In a third aspect, there is provided a surgical implant assembly comprising:
first and second anchor bodies adapted to engage respective bone surfaces;
a primary suture having a suture length extending between first and second terminal segments of the primary suture, the suture length of the primary suture further comprising:
   a first suture segment that passes through the first anchor body; and
   a second suture segment passing through the second anchor body with a bridging segment of the primary suture extending between the first and second suture segments of the primary suture
   wherein the first suture segment of the primary suture lies in between the bridging segment and the first terminal segment of the primary suture and wherein the second suture segment of the primary suture lies in between the bridging segment and the second terminal segment of the primary suture;
      a secondary suture having a suture length extending between first and second terminal segments of the secondary suture, the suture length of the secondary suture further comprising a first suture segment of the secondary suture that passes through the second anchor body wherein no part of the secondary suture passes through the first anchor body and wherein during use, the bridging segment of the primary suture is adapted to overlap with the secondary suture.

In another aspect, there is provided a method of performing surgery, the method comprising: providing a surgical implant assembly comprising:
first and second anchor bodies adapted to engage respective bone surfaces;
a primary suture having a suture length extending between first and second terminal segments of the primary suture, the suture length of the primary suture further comprising:
a first suture segment that passes through the first anchor body; and
a second suture segment passing through the second anchor body with a bridging segment of the primary suture extending between the first and second suture segments of the primary suture
wherein the first suture segment of the primary suture lies in between the bridging segment and the first terminal segment of the primary suture and wherein the second suture segment of the primary suture lies in between the bridging segment and the second terminal segment of the primary suture;
a secondary suture having a suture length extending between first and second terminal segments of the secondary suture, the suture length of the secondary suture further comprising a first suture segment of the secondary suture that passes through the second anchor body wherein no part of the secondary suture passes through the first anchor body and wherein during use, the bridging segment of the primary suture is adapted to overlap with the secondary suture
   inserting the first anchor body through a first bore drilled across a first bone region such that the first bore extends between proximal end and a distal end of the first bore such that the first anchor body is inserted into an opening at the proximal end and passed through the first bore to position the first anchor body at the distal end of the first bore;
   inserting the second anchor body though a second bore drilled across a second bone region such that the second bore extends between proximal end and a distal end of the bore such that the second anchor body is inserted into an opening at the proximal end of the second bore to position the second anchor body at the distal end of the second bore;
   tensioning the primary suture to effect engagement and initial anchoring of the first and second anchor bodies at the respective distal ends of the first and second bores resulting in the bridging segment of the primary suture to lie along portions of the first and second bone regions;
   tying the terminal ends of tensioned primary suture to form a primary knot;
   tensioning the secondary suture to effect further engagement and anchoring of the second anchor body at the respective distal end of the second bore;
   passing the terminal ends of the secondary suture through soft tissue adjoining the first and second bone regions; and
   tying the terminal ends of the secondary suture to form a secondary knot.

In a fourth aspect, the invention provides a surgical implant assembly comprising:
first and second anchor bodies adapted to engage respective bone surfaces;
a suture having a suture length extending between first and second terminal segments of the primary suture, the suture length further comprising: a leading segment and a return segment with a bridging segment being located between the leading segment and the return segment,
   wherein each of the leading segment and the return segment passes through the first anchor body; and
   wherein the bridging segment passes through the second anchor body to allow the second anchor body to be shuttled along the bridging segment during use.

In another aspect, there is provided a method of performing surgery, the method comprising: providing a surgical implant assembly comprising:
first and second anchor bodies adapted to engage respective bone surfaces;
a suture having a suture length extending between first and second terminal segments of the primary suture, the suture length further comprising: a leading segment and a return segment with a bridging segment being located between the leading segment and the return segment,
   wherein each of the leading segment and the return segment passes through the first anchor body; and
   wherein the bridging segment passes through the second anchor body to allow the second anchor body to be shuttled along the bridging segment during use;
   folding the suture implant assembly by moving the bridging segment and the second anchor body towards the terminal segments such that in a folded configuration, the first anchor body is located at one end and the second anchor body and terminal segments are located at an opposite end of the folded implant assembly;
   inserting and passing the first anchor body, in the folded configuration, through a first bore drilled across a first bone region such that the first bore extends between proximal end and a distal end of the first bore such that the first anchor body is inserted into an opening at the proximal end and passed through the first bore to position the first anchor body at the distal end of the first bore and passing the first anchor into a second bore drilled across a second bone region such that the second bore extends between proximal end and a distal end of the bore wherein the first anchor is positioned at the distal end of the second bore;
   locating the second anchor body, at least portions of the bridging segment and the terminal segments outside and adjacent the proximal end of the of the first bore;
   allowing the terminal ends of the suture to extend out of the opening of the first bore;
   tying the terminal ends of the suture to tension the suture to effect engagement and anchoring of the first and second anchor bodies at the respective first and second bores.

### BRIEF DESCRIPTION OF THE SEVERAL VIEWS OF THE DRAWING(S)

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a top perspective view of a surgical implant assembly 1000 in accordance with a first embodiment.

FIG. 2A is an enlarged view of the first anchor body 100A.

FIG. 2B is an enlarged view of the second anchor body 100B.

FIG. 2C is an enlarged view of the sleeve member 500 with primary and secondary sleeve portions 520 and 540.

FIGS. 3A to 3D show the perspective view from the Palmar side (front) of the right hand such that the thumb is to the right, referred to as the radial side or edge, and the index finger and other fingers are to the left, referred to as the ulnar side or edge.

FIGS. 3A to 3D sequentially illustrate insertion and deployment of the implant assembly 1000 in accordance with a method embodiment.

FIG. 4 is a perspective view of an inserter 2000 in accordance with a preferred embodiment.

FIG. 5 is an exploded view of the inserter 2000.

FIGS. 6 and 7 illustrate top and bottom views of the inserter 2000.

FIG. 8A is a top perspective view of a surgical implant assembly 2050 in accordance with a second embodiment.

FIGS. 8B and 8C illustrate steps for deployment of the implant assembly 2050.

FIG. 9A is a top perspective view of a surgical implant assembly 3000 in accordance with a third embodiment.

FIGS. 9B and 9C illustrate steps for deployment of the implant assembly 3000.

FIG. 10A is a top perspective view of a surgical implant assembly 4000 in accordance with a fourth embodiment.

FIGS. 10B and 10C illustrate steps for deployment of the implant assembly 4000.

FIG. 11A is a top perspective view of a surgical implant assembly 7000 in accordance with a fifth embodiment.

FIGS. 11B to 11D illustrate steps for deployment of the implant assembly 7000.

### DESCRIPTION OF SPECIFIC EMBODIMENTS OF THE INVENTION

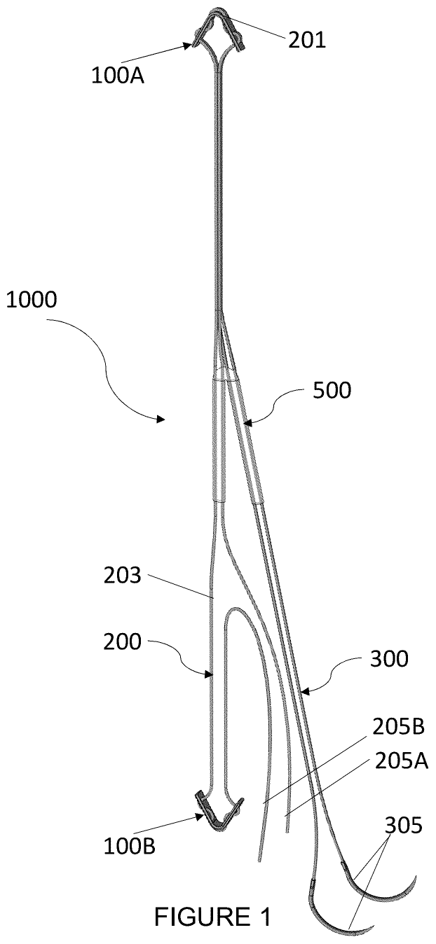

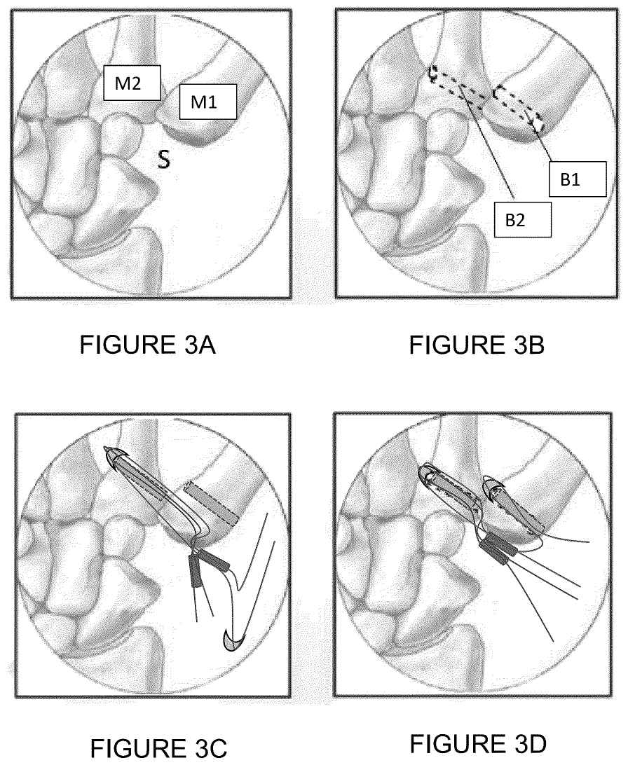

FIGS. 1 to 2A to 2C and 3A to 3D illustrate a surgically implantable sling assembly 1000 which is particularly well suited for carrying out trapeziectomy and suspensionplasty in which the trapezium (one of the carpal bones of the wrist that forms part of the thumb joint) is removed to manage symptoms of thumb arthritis. Thumb arthritis is also known as carpometacarpal joint osteoarthritis. The method steps for carrying out the suspensionplasty have been illustrated stepwise in FIGS. 3A to 3D and will be explained in the foregoing sections after the surgical implant assembly 1000 and its various key components have been described.

FIG. 1 illustrates an assembled view of the surgical implant assembly 1000 which consists of first and second flexible suture anchor bodies 100A and 100B that have a substantially identical configuration. The anchor bodies 100A and 100B are preferably made from flexible and biocompatible material. However, in other embodiments, the anchor bodies 100A and 100B may be made from non-flexible materials including but not limited to biocompatible alloys or hard polymeric buttons. Each of the anchor bodies generally denoted by 100 comprises a deformable and elongate configuration preferably made from fibrous material. The anchor bodies 100 are sufficiently deformable to allow the deformed anchor bodies to be pushed through bone tunnels drilled into metacarpal bones as will be discussed in the foregoing sections. The flat and fibrous construct of the anchor bodies 100 allows the anchor bodies to not only be deformable but also allows one or more sutures to be passed through the fibrous body of the anchor bodies through a plurality of apertures that allow suture to be passed through. These apertures may be formed by piercing the flat fibrous construct of the anchor bodies 100 at multiple locations. The flat and elongate profile of the anchor bodies 100 may be folded along a middle region of the anchor bodies 100 to allow the two ends of the anchor bodies to move closer and define a somewhat U-shaped or V-shaped configuration having an apex portion with anchor limbs extending from the apex portion. Such a configuration allows the apex portion of the anchor bodies 100 to be inserted into a drilled bone tunnel which results in the anchor limbs being moved towards each other into a radially narrow configuration while the anchor bodies 100 are pushed through bone tunnels.

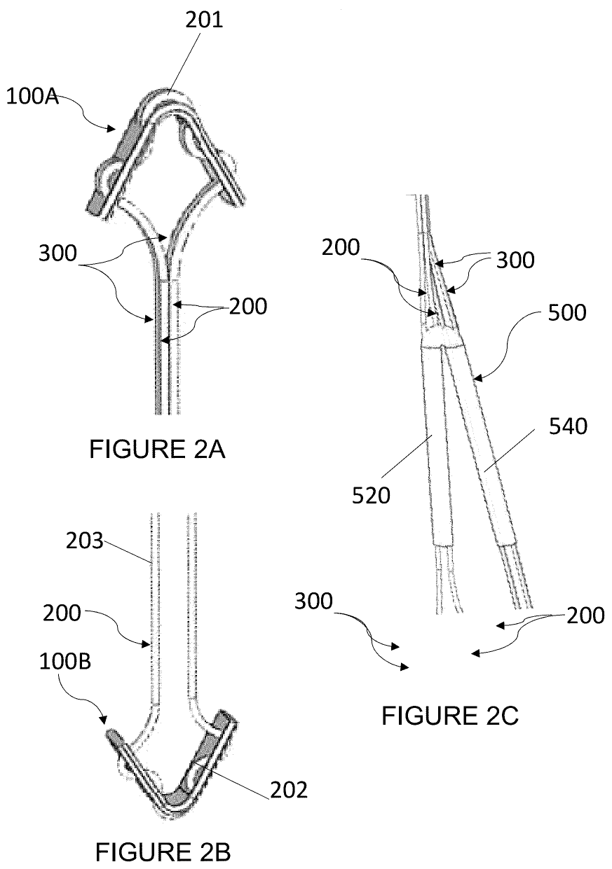

As shown most clearly in FIGS. 2A and 2B, each anchor body 100 and 200 comprises a flat and elongate profile with two limbs of the anchor extending from an apex portion of the anchor bodies. Each anchor body 100 comprises an outer surface 112 and an inner surface 114. One or more sutures are pierced into the inner surface 114 at a first location that is spaced away from one end of the anchor body 100 and passed through the thickness of the anchor body 100 resulting in the suture being effectively woven through the length of the anchor body 100 before extending out of a second spaced location that is spaced away from another end of the anchor body 100. Such a configuration of the anchor body 100 results in the anchor body 100 undergoing a radial expansion of the deformable anchor body 100 when the suture passing through the anchor body 100 is tensioned which results in the anchor bodies 100A or 100B engaging against bone tissue and no longer being sufficiently narrow to pass through the respective bone tunnels. It may be understood that other anchor body configurations that allow a soft and deformable anchor body such as 100 to be passed through a narrow bone tunnel followed by radial expansion when the suture(s) passing through the anchor body 100 is tensioned. As previously discussed, the deformable anchor bodies 100A and 100B are not limiting and non-deformable anchor bodies may also be used to achieve the intended function of the anchor bodies 100 in other alternative embodiments.

The first and second anchor bodies 100A and 100B are interconnected by a primary suture 200. The implant assembly 1000 also comprises a secondary suture 300 that also passes through the first anchor body 100A but does not pass through the second anchor body 100B. The secondary suture 300 comprises a length (the secondary suture length) that extends between terminal ends of the secondary suture 300. The terminal ends for the secondary suture 300 are provided with needles 305 that enable the sutures to be passed through tissue as will be discussed in the foregoing sections.

The primary suture 200 comprises a primary suture length that extends between the terminal segments 205 of the primary suture 200. The primary suture 200 comprises a first primary suture segment 201 that passes from a first end of the first anchor body 100A and out of the second end of the first anchor body 100A in a manner as described in the previous sections to enable first anchor body 100A to undergo radial expansion when the primary suture 200 is tensioned. The primary suture 200 also comprises a second suture segment 202 that passes from a first end of the second anchor body 1001B to a second end of the second anchor body 100B once again in a manner as described in the previous sections to enable first anchor body 100A to undergo radial expansion when the primary suture 200 is tensioned. A bridging segment bridging segment 203 extends from the second end of the first anchor body 100A to the first end of the second anchor body 1001B thereby resulting in primary suture passing through both the first and second anchor bodies 100A and 1001B. The first suture segment 201 lies in between the bridging segment 203 and the first terminal segment 205A and the second suture segment 202 lies in between the bridging segment 203 and the second terminal segment 205B. Optionally, a portion of the primary suture 200 that lies between the first suture segment 201 and the first terminal segment 205A may be optionally passed through the secondary anchor body 100B in an alternative embodiment.

Unlike the primary suture 200, the secondary suture 300 only passes through the first anchor body 100A. In the preferred embodiment, the secondary suture 300 also comprises terminal segments 305 with a secondary suture segment 301 (extending between the terminal segments 305) being passed through the primary anchor body 100A. Specifically, the secondary suture segment 301 follows a similar path as the first suture segment 201 of the primary suture 200 by extending into the first anchor body 100A at its first end and then being passed out of the second end of the first anchor body 100A through multiple apertures in the anchor body 100A to allow to enable first anchor body 100A to undergo radial expansion when the primary secondary suture 300 is tensioned.

As is evident from FIGS. 1 and 2A to 2C the implant assembly 100 uses a novel combination of two deformable anchors 100A and 100B in combination with the primary suture 200 (that connects the anchors 100A and 100B) while the secondary suture 300 is connected only to the first anchor body 1001B such that tensioning of the secondary suture 300 does not directly affect deformation or deployment of the second anchor body 100B. The working of the implant assembly 1000 will be discussed in detail in the foregoing sections.

Additionally, a primary suture sleeve portion 520 is provided for receiving and passing a first sleeve engaging segment of the primary suture 200 that extends from the first terminal segment 205A towards the first end of first anchor body 100A. The primary suture sleeve portion 520 also receives and passes a second sleeve engaging segment (that is part of the bridging segment 203) of the primary suture 200 that is returned from the second end of the anchor body 100A to extend towards the second anchor body 100B as has been previously described. The first sleeve portion 520 thus assists in forming a first loop of the primary suture 200 that engages the first anchor body 100A. The primary suture sleeve portion 520 is elongate and extends between first and second ends of the primary suture sleeve 520 such that the primary suture 200 is received into the first end of the primary suture sleeve 520 before being passed out from the second end of the primary suture sleeve 520 to pass the primary suture segment 201 into the first end of the anchor body 100A. The primary suture 200 is then returned back from the second end of the first anchor body 100A towards the second end of the primary suture sleeve 520 and then passed through the primary suture sleeve before exiting the primary suture sleeve 520 through its second end to extend towards the first end of the second anchor body 100B. As a result, the first terminal segment 205A extends out from the first end of the primary sleeve portion 520 while the second terminal segment 205B extends from the second end of the second anchor body 100B.

A secondary suture sleeve portion 540 is also provided for receiving and passing a first sleeve engaging segment of the secondary suture 300 that extends from the first terminal end towards the first end of the first anchor body 100A. The secondary sleeve portion 540 also receives a second sleeve engaging segment that is returned from the second end of the second anchor body 100B. As a result, the terminal segments 305 extend out from a first open end of the secondary suture sleeve portion 540.

In the preferred embodiment, the primary and secondary sleeve portions 520 and 540 are joined at a joining location along their second ends as illustrated most clearly in FIG. 2C. The primary and secondary sleeve portions 520 and 540 may be formed from a unitary hollow sleeve body made from a flexible and woven material by forming a large slit along a middle portion to divide the sleeve into the primary and secondary sleeve portions 520 and 540 that are joined at a joining location in the middle portion to allow the primary and secondary sleeve portions 520 and 540 to be movably disposed about the joining location to define a V-shaped configuration which allows their use as a supporting sling.

FIGS. 3A to 3D illustrate the perspective view from the Palmar side (front) of the right hand such that the thumb is to the right, referred to as the radial side or edge, and the index finger and other fingers are to the left, referred to as the ulnar side or edge. The implant assembly 100 which includes the anchor bodies 100 and the sling 500 exhibit rotational symmetry and hence can be implanted in either the right or left hand. All of the Figures show the trapezium bone being removed leaving behind an arthroplasty space S.

The arthroplasty procedure results in the sling 500 being anchored or tethered in place on the articular base of the thumb metacarpal using suture anchor bodies 100A and 100B, and secondary suture 300. The first anchor body 100A forms the leading anchor that is inserted through a bore B2 drilled into the index metacarpal (M2). A first bore B2 drilled into the index metacarpal extends between two openings located on index metacarpal. The leading suture anchor 100A is inserted through the bore B2 to anchor the leading suture anchor 100A on a metacarpal bone surface of the index metacarpal bone M2 as shown in FIG. 3C. A second bore B1 is drilled through the thumb metacarpal bone M1. The second bore B1 also extends between two openings within the thumb metacarpal bone which allows the second anchor body 100B to form a trailing anchor that is positioned to be anchored against a metacarpal bone surface of the thumb metacarpal bone M1. The anchoring of the suture anchors 100A results in the primary and secondary sutures 200 and 300 extending through the first bore B2 of the index metacarpal bone M2. The insertion of the second anchor body 100B through the second bore B1 of the thumb metacarpal bone M1 results in the second anchor body 100B being anchored against a metacarpal bone surface of the thumb metacarpal bone M1. The anchoring of the second anchor body 100B results in the primary suture 200 also passing through the second bore B1 as shown in FIG. 3D.

The needles located at the terminal segments 305 for the secondary suture 300 are used for piercing the Abductor Pollicis Longus tendon to secure the secondary suture 300 with the tendon. This step is accompanied by tensioning of the secondary suture 300 that results in the radial expansion of the first anchor body 100A thereby resulting in the first anchor body 100A being secured against the bone surface of the index metacarpal M2 while the sleeve 500, particularly sleeve portions 520 and 540 are positioned along articular base of the thumb metacarpal. Specifically, the positioning and securement of the second anchor body 100B against the metacarpal surface of the thumb metacarpal bone M1 results in the sleeve portions 520 and 540 being positioned to function as a sling that supports the articular base of the thumb metacarpal. The terminal segments 205 may be tied with each other to form a knot once the primary suture 200 has also been adequately tensioned to secure the second anchor body 100B. Fully tensioning the primary suture 200 allows the sleeve 500 (with sleeve portions 520 and 540) to function as a basal joint stabilizer that is tethered or anchored by the anchor bodies 100A and 1001B. Tensioning of the primary suture 200 by pulling the terminal ends 250 also conforms the shape of the sleeve portions 520 and 540 with the complex shape of the articular base of the thumb metacarpal bone M1.

It would also be understood that the sleeve 500 is optional and in at least some alternative embodiments. As evident from the accompanying Figures, the sleeve portions 520 and 540 surround the primary and secondary sleeve engaging elements of the primary and secondary sutures 200 and 300 respectively. Whilst the provision of the sleeve portions 520 540 provides additional surface area for support, the primary function of supporting or stabilizing the basal joint can be carried out by portions of the primary and secondary sutures 200 and 300 that are positioned along the basal joint as a result of the novel configuration of the aforementioned anchor bodies 100A, 100B and the primary and secondary sutures 200 and 300 in the surgical implant sling assembly 1000.

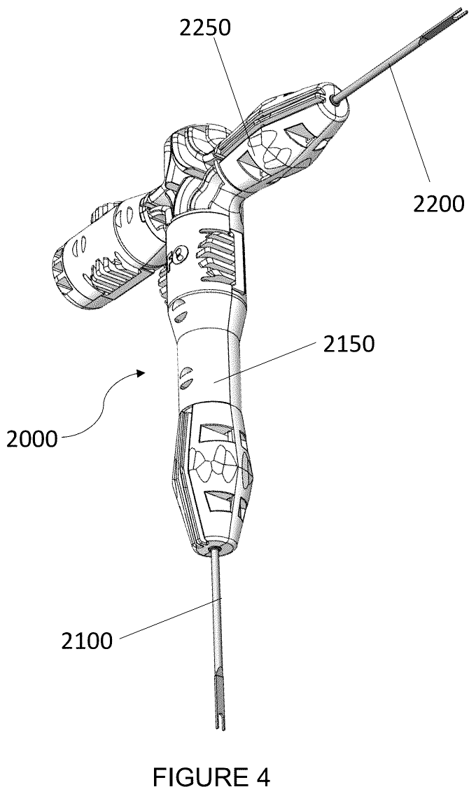

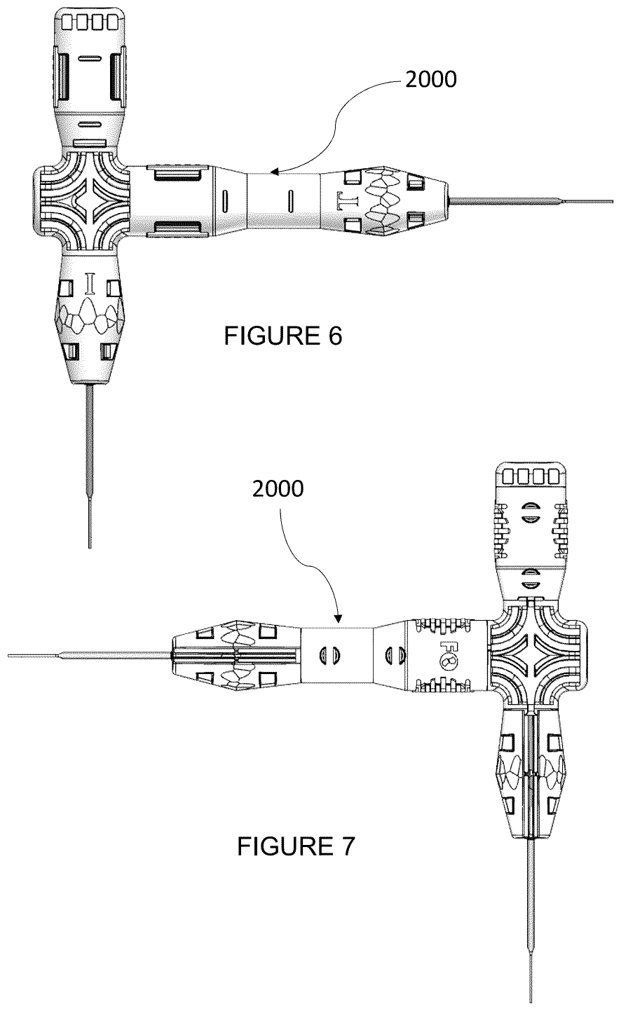

FIGS. 4 to 6 illustrate an inserter 2000 which is particularly (but not exclusively) well suited for deployment of the implant assembly 1000 described in the foregoing sections. Whilst the features of the inserter 200 have been described with reference to the implant assembly 1000, the scope of this inserter 2000 is not limited to the specific configuration of the implant assembly 1000 and other implant assemblies with multiple anchor bodies (such as anchor bodies 100A and 100B) may be used without departing from the spirit and scope of the inserter 2000. The inserter 2000 comprises a first and second elongate shafts 2100 and 2200 that are mutually perpendicular to each other. Each of the shafts 2100 and 2200 is identical in the preferred embodiment. Each shaft (2100 or 2200) comprises a pair of spaced apart tines positioned at a distal end of the shaft to define a bone tunnel engaging portion for being pushed into the metacarpal bones M1 or M2 to therein create the respective bone tunnels. The tines for each of the shafts (2100 and 2200) are spaced apart to define a slot therebetween that forms an anchor carrying portion that is immediately proximal to the bone tunnel forming portion.

Elongate shaft 2200 is used for insertion and loading of the first anchor body 100A. Specifically, the first anchor body 100A is positioned in the anchor carrying portion of the spaced apart tines located at the distal end of the first elongate shaft 2200 and segments of the primary and secondary sutures 200 and 300 extend from the proximal end of the first elongate shaft 2200 towards the anchor carrying portion before returning back from the distal end towards the proximal end of the elongate shaft 2200. The second anchor body 100B is positioned in the anchor carrying portion of the spaced apart tines located at the distal end of the second elongate shaft 2100 and segments of the primary suture 200 extend from the proximal end of the second elongate shaft 2100 towards the anchor carrying portion before returning back from the distal end towards the proximal end of the second elongate shaft 2100. Each of the proximal portions of the shafts 2100 and 2200 is coupled with an inserter body with respective housing portions 2150 (2150A and 2150B) and 2250 (2250A and 2250B) that are also mutually perpendicular to each other. Each of the housing portions 2150 and 2250 is substantially hollow and provides channels for winding the primary and secondary sutures 200 and 300 therein. The inserter allows for each shaft 2100 and 2200 to be inserted through the bone tunnels B2 and B1 in order to position the anchor bodies 100A and 100B against the metacarpal bone surface of the index metacarpal M1 and the thumb metacarpal M2. Advantageously, the distal or leading end of each shaft 2100 and 2200 where the respective anchor bodies 100A and 100B are positioned comprises an overall thickness that is less than the thickness of the trailing portion of the shaft (2100 or 2200). As a result, when the anchor bodies 100A and 100B are loaded on the anchor receiving end portions of the shaft (2100 or 2200), the leading end of the shaft achieves a similar overall thickness when compared to the trailing portion of each shaft 2100 and 2200. Such a configuration further facilitates insertion of the anchor bodies 100A and 100B through the bone tunnels B1 and B2.

The first housing portion 2150 is provided with a first visual indicia such as the letter “I” to indicate that the first shaft 2100 should be inserted into the index metacarpal M1. The second housing position 2250 is also provided with a second visual indicia such as the letter “T” to indicate that the second shaft 2100 should be inserted into the thumb metacarpal bone M2.

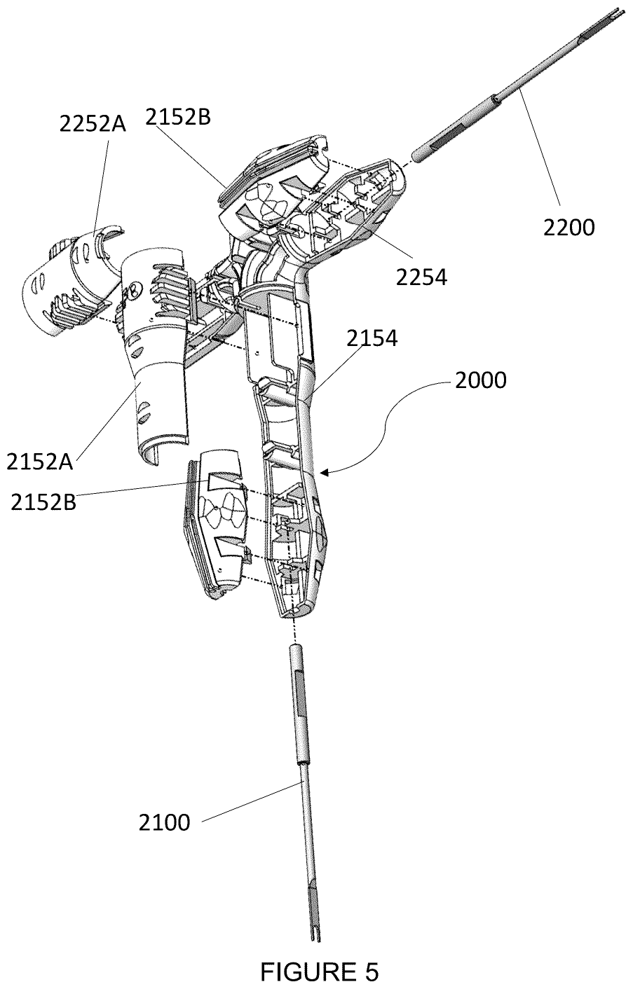

As shown best in FIG. 5, the housing body 2150 may comprise cover portion 2152A to easily access the primary and secondary sutures 200 and 300 wound along winding channels provided on a holding portion 2154 of the housing body 2150. Similarly, the housing body 2250 may also comprise removable cover portions 2252A to easily access the primary suture 200 wound along winding channels provided on a holding portion 2254 of the housing body 2250.

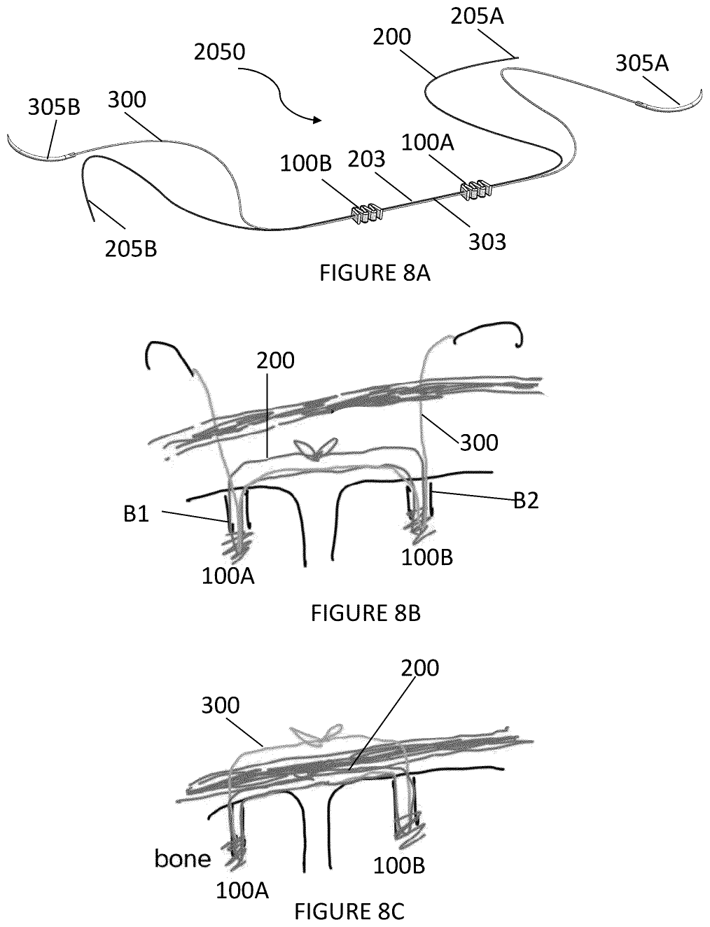

FIGS. 8A to 8C illustrate another embodiment of a surgical implant assembly 2050 is illustrated. Like reference numerals denote like features as previously described in the preceding sections. The surgical implant assembly 2000 also consists of first and second flexible suture anchor bodies 100A and 100B that are preferably made from flexible and biocompatible material. However, in other embodiments, the anchor bodies 100A and 100B may be made from non-flexible materials as has been previously described in the preceding sections.

Implant assembly 2050 comprises a primary suture 200 having a suture length extending between first and second terminal segments 205A and 205B. The primary suture 200 comprises a first suture segment that passes through the first anchor body 100A and a second suture segment passing through the second anchor body 100B with a bridging segment 203 of the primary suture 200 extending between the first and second suture segments (and first and second anchors 100A and 100B) of the primary suture 200.

The first suture segment of the primary suture 200 lies in between the bridging segment 203 and the first terminal segment 205A of the primary suture 200 and the second suture segment of the primary suture 200 lies in between the bridging segment 203 and the second terminal segment 205B of the primary suture 200.

The surgical implant assembly 2050 also comprises a secondary suture 300 having a suture length extending between first and second terminal segments 305A and 305B of the secondary suture 300. The suture length of the secondary suture 300 also comprises a first suture segment that passes through the first anchor body 100A; and a second suture segment passing through the second anchor body 100B with a bridging segment 303 of the secondary suture 300 extending between the first and second suture segments of the primary suture 300. As shown in the FIG. 8A, the first suture segment of the secondary suture 300 lies in between the bridging segment 303 and the first terminal segment 305A of the secondary suture 300 and wherein the second suture segment of the secondary suture 300 lies in between the bridging segment 303 and the second terminal segment 305B of the secondary suture 300. As will be evident from the foregoing description of use, the bridging segments 203 and 303 of the primary and secondary sutures 200 and 300 are adapted to overlap with each other.

Referring to FIGS. 8B, a method of performing surgery using the surgical implant 2050 is illustrated. The method comprises an initial step of inserting the first anchor body 100A through a first bore B1 drilled across a first bone region such that the first bore B1 extends between proximal end and a distal end of the first bore B1 such that the first anchor body 100A is inserted into an opening at the proximal end and passed through the first bore to position the first anchor body at the distal end of the first bore.

Similarly, the second anchor body 1001B though a second bore B2 drilled across a second bone region such that the second bore B2 extends between proximal end and a distal end of the second bore b2 such that the second anchor body 100B is also inserted into an opening at the proximal end of the second bore B2 to position the second anchor body 1001B at the distal end of the second bore.

Once the first and second anchor bodies 100A and 1001B have been positioned at the distal ends of the first and second bores B1 and B2, the subsequent step involves tensioning the primary suture 200 to effect engagement and initial anchoring of the first and second anchor bodies 100A and 1001B at the respective distal ends of the first and second bores B1 and B2 resulting in the bridging segment 203 of the primary suture 200 to overlap with the bridging segment 303 of the secondary suture 300 and lie along portions of the first and second bone regions. Once the tensioning of the primary suture 200 has been undertaken, the terminal segments 205A and 205B are tied to form a first knot to maintain the first suture 200 in a tensioned configuration.

Once the first knot has been formed, the next step involves tensioning the secondary suture 300 to effect further engagement and anchoring of the first and second anchor bodies 100A and 100B at the respective distal ends of the first and second bores B1 and B2. The terminal ends 305A and 305B of the secondary suture 300 comprises piercing needles for allowing the secondary suture 300 to enable the secondary suture 300 to be passed through soft tissue adjoining the first and second bone regions. Once the needles of the secondary suture 300 have been passed through the soft tissue, the terminal ends 305A and 305B are tied into a second knot to maintain the secondary suture 300 in a tensioned configuration for holding the soft tissue to the first and second bone regions.

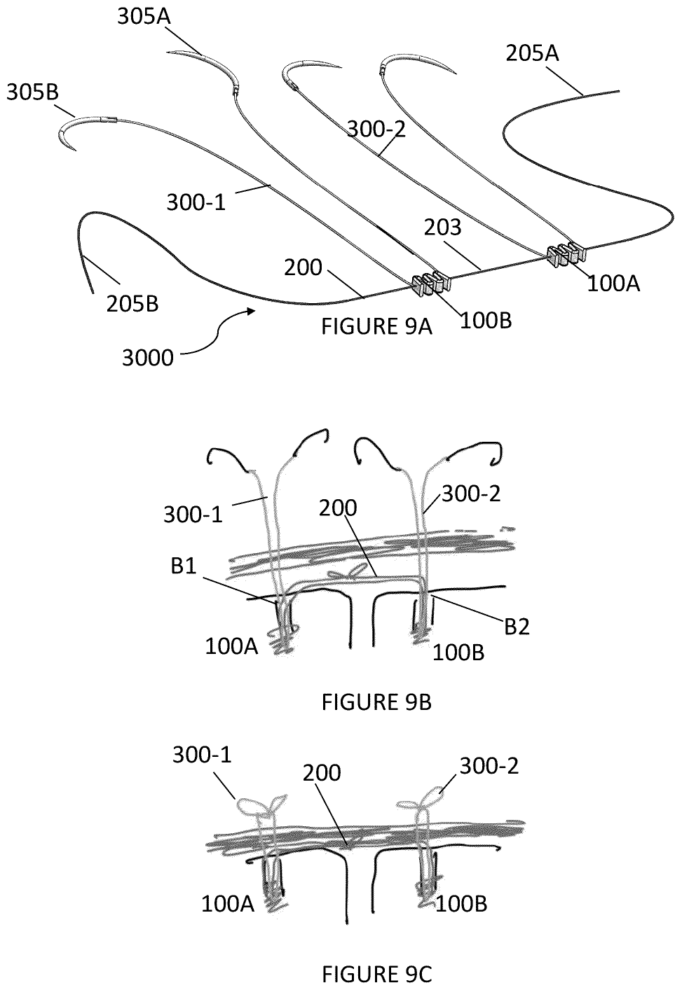

Referring to FIGS. 9A to 9C, another embodiment of a surgical implant assembly 3000 is illustrated. Like reference numerals denote like features as previously described in the preceding sections. The surgical implant assembly 3000 also consists of first and second flexible suture anchor bodies 100A and 100B that are preferably made from flexible and biocompatible material. However, in other embodiments, the anchor bodies 100A and 100B may be made from non-flexible materials as has been previously described in the preceding sections.

The surgical implant assembly 3000 comprises the first and second anchor bodies 100A and 100B in combination with a primary suture 200 and two secondary sutures 300-1 and 300-2. Once again, the primary suture 200 comprises a suture length extending between first and second terminal segments 205A and 205B of the primary suture 200. The suture length of the primary suture 200 comprises a first suture segment that passes through the first anchor body 100A and a second suture segment passing through the second anchor body 100B. A bridging segment 203 of the primary suture 200 extends between the first and second suture segments of the primary suture 200. The first suture segment of the primary suture 200 lies in between the bridging segment 203 and the first terminal segment 205A of the primary suture 200 and the second suture segment of the primary suture 200 lies in between the bridging segment 203 and the second terminal segment 205B of the primary suture 200.

The surgical implant assembly 3000 further includes two additional secondary sutures 300-1 and 300-2 which engage the first and second anchor bodies 100A and 1001B respectively. Each of the secondary sutures (generally denoted by 300) comprises a suture length with terminal ends 305-A and 3055-B respectively. As shown most clearly in FIG. 9A, the first secondary suture 300-1 passes through the first anchor body 100A such that no section of the first secondary suture 300-1 passes through the second anchor body 100B. Similarly, the second secondary suture 300-2 passes through the second anchor body 100B such that no section of the second secondary suture 300-2 passes through the first anchor body 100A. In short, each of the secondary sutures 300-1 and 300-2 is exclusively engaged with a respective anchor body 100A and 100B.

FIGS. 9B and 9C illustrate a method of performing surgery using the surgical implant assembly 3000.

The first step involves inserting the first anchor 100A body through a first bore B1 drilled across a first bone region such that the first bore extends between proximal end and a distal end of the first bore B1 such that the first anchor body is inserted into an opening at the proximal end and passed through the first bore B1 to position the first anchor body 100A at the distal end of the first bore B1.

Similarly, the method also involves inserting the second anchor body 1001B though a second bore B2 drilled across a second bone region such that the second bore b2 extends between proximal end and a distal end of the bore b2 such that the second anchor body 100B is inserted into an opening at the proximal end of the second bore B2 to position the second anchor body 1001B at the distal end of the second bore B2.

Once the two anchors 100A and 100B have been positioned at the respective distal ends of the bores B1 and B2, the next step involves tensioning the primary suture 200 to effect engagement and initial anchoring of the first and second anchor bodies 100A and 1001B at the respective distal ends of the first and second bores B1 and B2 resulting in the bridging segment 203 of the primary suture 200 to lie along portions of the first and second bone regions as shown clearly in FIG. 9B. tensioning of the primary suture 200 is followed by tying the terminal ends 205A and 205B of the tensioned primary suture 200 to form a primary knot and maintain the primary suture 200 in a tensioned configuration.

The next step involves tensioning the first and second secondary sutures 300-1 and 300-2 to effect further engagement and anchoring of the first and second anchor bodies 100A and 100B at the respective distal end of the first and second bores and passing the terminal ends of the first and second secondary sutures 300-1 and 300-2 through soft tissue adjoining the first and second bone regions. The terminal ends of the secondary sutures 300-1 and 300-2 are provided with needles to allow the needles to pierce through the soft tissue thereby allowing the secondary sutures 300-1 and 300-2 to hold soft tissue against the first and second bond regions once the terminal ends of the tensioned first and second secondary sutures are formed into two corresponding secondary knots.

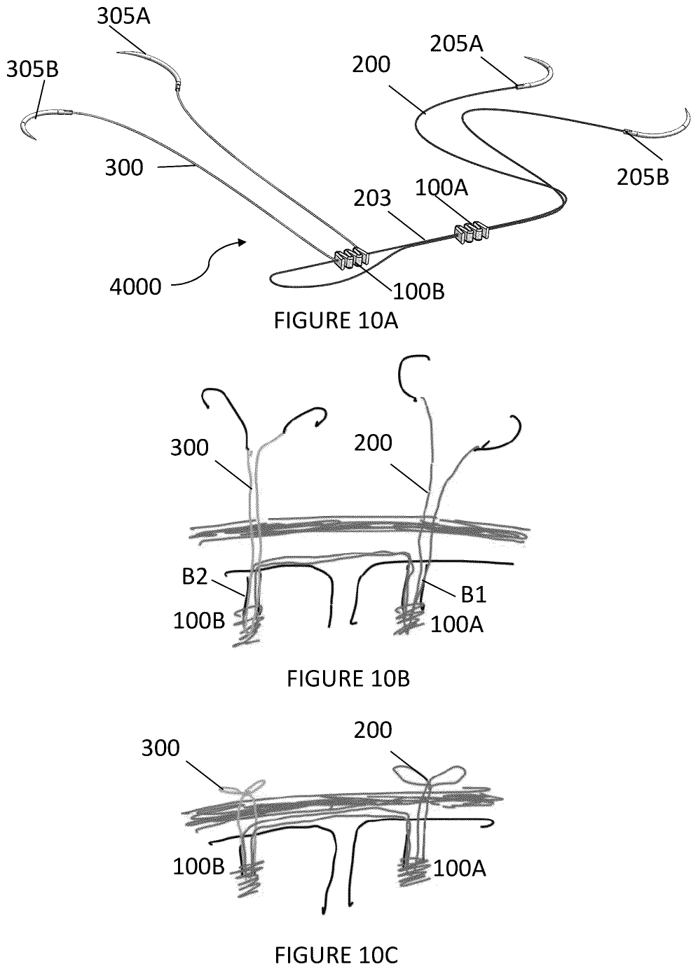

Referring to FIGS. 10A to 10C, another embodiment of a surgical implant assembly 4000 is illustrated. Like reference numerals denote like features as previously described in the preceding sections. The surgical implant assembly 3000 also consists of first and second flexible suture anchor bodies 100A and 1001B that are preferably made from flexible and biocompatible material. However, in other embodiments, the anchor bodies 100A and 100B may be made from non-flexible materials as has been previously described in the preceding sections.

The surgical implant assembly 4000 comprises a primary suture 200 having a suture length extending between first and second terminal segments 205A and 205B of the primary suture 200. The primary suture 200 comprises a first suture segment that passes through the first anchor body 100A; and a second suture segment passing through the second anchor body 100B with a bridging segment 203 of the primary suture 200 extending between the first and second suture segments of the primary suture 200. The first suture segment of the primary suture 200 lies in between the bridging segment 203 and the first terminal segment 205A of the primary suture 200 and wherein the second suture segment of the primary suture 200 lies in between the bridging segment 203 and the second terminal segment 205B of the primary suture 200.

The surgical implant assembly 4000 also comprises a secondary suture 300 having a suture length extending between first and second terminal segments 305A and 305B of the secondary suture 300. The suture length of the secondary suture 300 further comprising a first suture segment of the secondary suture 300 that passes through the second anchor body 100B and no part of the secondary suture 300 passes through the first anchor body 100A. As will be evident from the foregoing description of use, the bridging segment 203 of the primary suture 200 is adapted to overlap with the secondary suture 300.

FIGS. 10B and 10C illustrate a method of performing surgery using the surgical implant assembly 4000.

The method comprises inserting the first anchor body 100A through a first bore B1 drilled across a first bone region such that the first bore B1 extends between proximal end and a distal end of the first bore B1 such that the first anchor body 100A is inserted into an opening at the proximal end and passed through the first bore B1 to position the first anchor body 100A at the distal end of the first bore B1.

Similarly, the second anchor body 1001B is inserted through a second bore B2 drilled across a second bone region such that the second bore B2 extends between proximal end and a distal end of the bore such that the second anchor body 1001B is inserted into an opening at the proximal end of the second bore B2 to position the second anchor body 100B at the distal end of the second bore B2.

The method comprises tensioning the primary suture 200 to effect engagement and initial anchoring of the first and second anchor bodies 100A and 100B at the respective distal ends of the first and second bores B1 and B2 resulting in the bridging segment 203 of the primary suture 200 to lie along portions of the first and second bone regions. Once the primary suture 200 is tensioned, the terminal ends of the primary suture 200 are passed through soft tissue (by way of piercing the soft tissue with the piercing needles at the end of the terminal segments of the primary suture 200) tied to form a primary knot.

Once the primary suture 200 has been tensioned and the primary knot has been formed, the next step involves tensioning the secondary suture 300 to effect further engagement and anchoring of the second anchor body 1001B at the respective distal end of the second bore B2. The terminal ends of the secondary suture 300 are also passed through the soft tissue adjoining the first and second bone regions followed by tying the terminal ends of the secondary suture 300 to form a secondary knot.

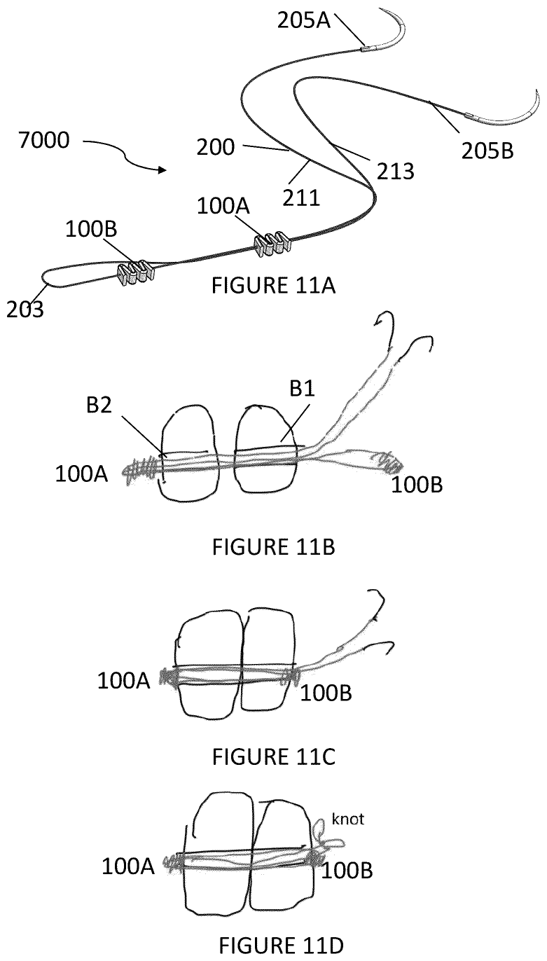

Referring to FIGS. 11A to 11D, yet another embodiment of the surgical implant assembly 7000 is illustrated. Like reference numerals denote like features as previously described in the preceding sections. The surgical implant assembly 3000 also consists of flexible suture anchor bodies 100A and 100B that are preferably made from flexible and biocompatible material. The anchor bodies 100 may be made from non-flexible materials as has been previously described in the preceding sections.

The surgical implant assembly 7000 comprises a suture 200 having a suture length extending between first and second terminal segments 205A and 205B of the primary suture 200. The suture length further comprises: a leading segment 211 and a return segment 213 with a bridging segment 203 being located between the leading segment 211 and the return segment 213.

FIGS. 11B to 11D, illustrate a surgical method for performing surgery by utilizing the surgical implant assembly 7000.

As shown in FIGS. 11B to 11D, two bones adjacently arranged bones may be anchored to each other which may result in the two bones being brought closer to each other by utilizing the surgical implant assembly 7000. A thorough continuous bore may be drilled through the bones such that bore portion B1 passes through the first bone and bore portion B2 passes through the second bone to form a generally continuous bore B when the implant 7000 is used for drawing the bones closer to each other. The initial step involves folding the implant assembly 7000 in half as shown in FIG. 11B and the distal portion of the folded surgical implant assembly 7000 is inserted into the proximal end opening for the first bore B1 before being passed into the second bore portion B2 to position the first anchor body 100A at the distal end of the continuous bore B. It is important to note that the terminal segments 205A and 205B are to extend out of the proximal end opening and should not be inserted into the first bore region B1. It is also important to note that the second anchor body 1001B should continue to be located outside the continuous bore B and should preferably be located outside and adjacent the proximal end opening thereby resulting in the bridging segment 203 being located at the proximal end opening for the bore B. This may be referred to as the initial positioning step.

The initial positioning step is followed by a tensioning step whereby the terminal segments 205A and 205B are tensioned which results in anchor bodies 100A and 100B expanding that prevents the anchors from passing through the bore regions B2 and B1 respectively even upon tensioning the terminal segments of the suture. As a result, any tensioning of the suture results in the bones and the bore regions B1 and B2 being drawn towards each other to form the continuous bore region.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. A surgical implant assembly comprising:
first and second anchor bodies adapted to engage respective bone surfaces;
a primary suture having a suture length extending between first and second terminal segments of the primary suture, the suture length of the primary suture further comprising:
a first suture segment that passes through the first anchor body; and
a second suture segment passing through the second anchor body with a bridging segment of the primary suture extending between the first and second suture segments of the primary suture
wherein the first suture segment of the primary suture lies in between the bridging segment and the first terminal segment of the primary suture and wherein the second suture segment of the primary suture lies in between the bridging segment and the second terminal segment of the primary suture;
a secondary suture having a suture length extending between first and second terminal segments of the secondary suture, the suture length of the secondary suture further comprising:
a first suture segment that passes through the first anchor body; and
a second suture segment passing through the second anchor body with a bridging segment of the secondary suture extending between the first and second suture segments of the primary suture
wherein the first suture segment of the secondary suture lies in between the bridging segment and the first terminal segment of the secondary suture and wherein the second suture segment of the secondary suture lies in between the bridging segment and the second terminal segment of the secondary suture; and
wherein during use, the bridging segment of the primary suture is adapted to overlap with the bridging segment of the secondary suture.

2. A surgical implant assembly in accordance with claim 1, wherein the first and second anchor bodies are sufficiently deformable to be passed through respective bone tunnels drilled in the first and second metacarpal bones.

3. A surgical implant assembly in accordance with claim 1, wherein the first suture segment of the primary and secondary sutures passes through a plurality of apertures in the first anchor body and the second suture segment of the primary and secondary sutures passes through a plurality of apertures in the second anchor body.

4. A surgical implant assembly in accordance with claim 1, wherein each of the first and second anchor bodies comprises an elongate and deformable structure with the primary and secondary sutures being pierced through the elongate and deformable structure along a plurality of piercing locations such that tensioning of the primary and/or secondary sutures increases radial dimensions of the first and second anchors to result in engagement of the first and second anchors with the respective bone surfaces.

5. A surgical implant assembly in accordance with claim 1, wherein each of the first and second anchor bodies comprises a flat and fibrous construct and wherein tensioning of the primary and/or secondary sutures results in the first and second anchor bodies to become bunched up.

6. A surgical implant assembly in accordance with claim 1, wherein terminal ends for the primary or secondary suture comprises needles for piercing through tissue and allowing the terminal ends to be passed through tissue.

7. A surgical implant assembly comprising:
first and second anchor bodies adapted to engage respective bone surfaces;
a suture having a suture length extending between first and second terminal segments of the primary suture, the suture length further comprising: a leading segment and a return segment with a bridging segment being located between the leading segment and the return segment,
wherein each of the leading segment and the return segment passes through the first anchor body; and
wherein the bridging segment passes through the second anchor body to allow the second anchor body to be shuttled along the bridging segment during use.

8. A surgical implant assembly in accordance with claim 7, wherein the first and second anchor bodies are sufficiently deformable to be passed through respective bone tunnels drilled in the first and second metacarpal bones.

9. A surgical implant assembly in accordance with claim 7, wherein the suture passes through a plurality of apertures in the first and second anchor bodies.

10. A surgical implant assembly in accordance with claim 7, wherein each of the first and second anchor bodies comprises an elongate and deformable structure with the primary and secondary sutures being pierced through the elongate and deformable structure along a plurality of piercing locations such that tensioning of the primary and/or secondary sutures increases radial dimensions of the first and second anchors to result in engagement of the first and second anchors with the respective bone surfaces.

11. A surgical implant assembly in accordance with claim 7, wherein each of the first and second anchor bodies comprises a flat and fibrous construct and wherein tensioning of the primary and/or secondary sutures results in the first and second anchor bodies to become bunched up.

12. A surgical implant assembly in accordance with claim 7, wherein terminal ends for the suture comprise needles for piercing through tissue and allowing the terminal ends to be passed through tissue.
